(19) United States
(12) Patent Application Publication
(10) Pub. No.: US 2023/0355891 A1
(43) Pub. Date: Nov. 9, 2023

(54) NEEDLE OUTPUT MECHANISM

(71) Applicant: CC Biotechnology Corporation, Tainan City (TW)

(72) Inventors: Chin-Min Yeh, Tainan City (TW); Pin-Han Wu, Tainan City (TW)

(21) Appl. No.: 18/028,508

(22) PCT Filed: Sep. 25, 2020
(86) PCT No.: PCT/CN2020/117744

§ 371 Date: Mar. 24, 2023

(51) Int. Cl. A61M 5/32

(52) U.S. Cl. CPC A61M 5/322

(57) ABSTRACT

A needle output mechanism which can be connected to a pharmaceutical agent container and a drive module. The base of the needle output mechanism is equipped with a movable connecting member, a needle set, a needle output operation assembly, and a needle retraction operation member. The connecting member is connected to an adapter mechanism. During injection, the needle set in the connecting member can be driven by the needle output operation assembly, and using a hard needle disposed passing through a soft needle and out from the base, outputs a needle which can directly puncture the human body, successively driving the hard needle (133) to disengage from the soft needle and inject the pharmaceutical agent solution by way of the drive module. After injection, the needle retraction operation member can retract the connecting member and the soft needle thereby facilitating operation.

### BACKGROUND OF THE INVENTION

### 1. Field of the Invention

The present invention relates to an injector used for subcutaneous injections of a pharmaceutical agent into a human body, especially to a needle output mechanism in the injector.

### 2. Description of the Prior Arts

A conventional injector used for subcutaneous injection of pharmaceutical agent into a human body has a manually or electrically operated injection module connected to a pharmaceutical agent container through a needle output mechanism, so a needle of the needle output mechanism can pierce a human body for subcutaneous injections.

In order to temporarily leave the needle in the human body so as to prevent discomfort of the human body caused by piercing and removal of the needle many times when multiple subcutaneous injections are to be performed on the human body, in the hope that the human body would not be injured by the needle indwelling in the human body, the needle of the conventional injector is substituted for a needle set that has a flexible soft needle and a hard needle. Since the soft needle is soft and is difficult to directly pierce the human body, the soft needle pierces the human body with the hard needle mounted inside the soft needle. Afterward the hard needle is drawn out from the soft needle and the soft needle is connected to the injection module and the pharmaceutical agent container for injections. The soft needle is able to stay longer in the human body for multiple injections.

However, since the needle set of the needle output mechanism of the conventional injector has the soft needle and the hard needle, an operator has to set up the needle with two steps, which is inconvenient and needs to be further improved.

To overcome the shortcomings, the present invention provides a needle output mechanism to mitigate or obviate the aforementioned problems.

### SUMMARY OF THE INVENTION

The main objective of the present invention is to provide a needle output mechanism that solves the problem of the inconvenient operation of setting up the needle.

The needle output mechanism has a base, a connecting member, a needle set, a needle output operation assembly, and a needle retraction operation member. The connecting member is moveably mounted in the base. The needle set is moveably mounted in the connecting member and has a soft needle, a needle plug, and a hard needle. The needle plug is mounted on an end of the soft needle. The hard needle is capable of being mounted in the soft needle. The needle output operation assembly is moveably mounted in the base and is connected to the needle set. The needle output operation assembly is capable of being operated to drive the needle set located in the connecting member to protrude out of the base with the hard needle mounted in the soft needle, and then drive the hard needle to detach from the soft needle. The needle retraction operation member is moveably mounted in the base. The 23 needle retraction operation member is capable of being operated to drive the connecting member and the soft needle in a needle-output state to retract back to the base.

The advantages of the present invention are as follows. The needle output mechanism is adapted to be assembled with a pharmaceutical agent container and a manual or electrical drive module to form an injector. During injection to a human body by the injector, the protruding operating assembly is operated to drive the needle set located in the connecting member, and makes the needle set protrude from the base with the hard needle mounted in the soft needle, such that the soft needle of the needle set is capable of piercing into the human body. During the operation applied on the needle output operation assembly, the hard needle is then driven to detach from the soft needle, and then the drive module drives the driving piston assembly of the adapter mechanism to run pharmaceutical agent injection. After injection, the needle retraction operation member is operated to retract the soft needle. As a result, the needle output mechanism of the present invention facilitates ease in operation.

Further, the base has an action room. Aback surface of the base has a base board and a penetration hole formed on the base board and connected to the action room. A side of the base has an action opening connected to the action room. A fixing buckling hook is mounted on the base and is located outside the action room. The connecting member is moveably mounted in the action room of the base. The connecting member has a connecting base segment and a connecting head. The connecting base segment has a base annular wall and a needle set mounting segment mounted in the base annular wall. The needle set mounting segment has a needle room formed in the needle set mounting segment and a needle plug hole formed on an end, which faces toward the base board, of the needle set mounting segment and connected to the needle room. The needle plug hole corresponds to the penetration hole of the base board. The connecting head is mounted on the connecting base and is connected to the needle room in the needle set mounting segment. The connecting head is mounted through the action opening of the base. The base annular wall has at least one cutting blade segment. The soft needle of the needle set is moveably mounted in the needle room of the needle set mounting segment. The needle plug that is mounted on the soft needle is capable of being fixed in the needle plug hole. The needle output operation assembly has a needle output operating unit, a needle base, a sealing unit, and an elastic unit. The needle output operating unit is moveably mounted in the base, and the needle output operating unit is capable of being fixed and buckled by the fixing buckling hook of the base. The needle output operating unit has a button segment and an accommodating room located on a side of the button segment. The accommodating room corresponds to the action room. The needle base is located in the accommodating room of the needle output operating unit and is connected to the needle output operating unit. The needle base has a needle base head end and a needle base connecting rod. A connecting segment is mounted between a periphery of the needle base head end and an inner wall of the accommodating room and is capable of being cut by the cutting blade segment. The needle base connecting rod is connected to the hard needle and is capable of pushing the needle plug of the needle set. The sealing unit assembled with the elastic unit is located in the connecting member. The sealing unit is located outside of the needle set mounting segment and covers the needle room. The sealing unit is tightly sleeved on the outer side of the needle base connecting rod. The elastic unit is sleeved on an outer side of the needle base connecting rod. Two ends of the elastic unit are respectively connected to the needle base head end of the needle base and the sealing unit. By this, in one single press applied on the needle output mechanism, the needle set protrudes from the base with the hard needle mounted in the soft needle. Additionally, the cutting blade segment of the connecting member cuts the connecting segment between the needle base and the needle output operating unit, and the elastic unit pushes the needle base to drive the hard needle to detach from the soft needle, so the soft needle is connected to the output adapter via the needle room and the connecting head, which facilitates ease in the operation of protruding the needle set.

Further, a diameter reducing segment is formed on an end, which is connected to the connecting base segment, of the connecting head of the connecting member, is formed with a reduced diameter and is capable of being cut off. A cutting knife segment is mounted on a side, which corresponds to the connecting head, of the needle output operating unit. The cutting knife segment is capable of being operated to cut off the diameter reducing segment of the connecting head of the connecting member. By this, when the abutting inclined surface of the needle retraction operation member of the needle output mechanism pushes the base board located on the back, which is away from the base, of the connecting member located in the base, the abutting inclined surface synchronously drives the connecting member and the soft needle mounted in the connecting member to retract to the base. Besides, the cutting knife segment of the needle output operating unit fixed on the base cuts the diameter reducing segment of the connecting head of the connecting member connected to the adapter mechanism, such that the needle output mechanism cannot be reused, thereby ensuring the safety in use.

The needle output mechanism may additionally have a safety cover and a safety bolt. The safety cover covers an outer side of the needle output operation assembly and is capable of engaging with the base. The safety bolt is mounted in the safety cover and the needle output operating unit of the needle output operation assembly. A pushing elastic unit is mounted between the safety cover and the needle output operating unit. Therefore, the needle output mechanism can be operated to protrude the needle only after the safety bolt is pulled out and the safety cover is detached, thereby ensuring safety in use. Besides, after the safety bolt is pulled out, the pushing elastic unit automatically pushes the safety cover away from the base, thereby enhancing operating convenience.

### BRIEF DESCRIPTION OF THE DRAWINGS

FIG. 1 is a perspective view of a preferred embodiment of a needle output mechanism in accordance with the present invention;

FIG. 2 is a front view of the preferred embodiment of the needle output mechanism in FIG. 1;

FIG. 3 is a perspective view of the preferred embodiment of the needle output mechanism in FIG. 1, showing the needle output mechanism applied in an electrical injector;

FIG. 4 is an exploded perspective view of the preferred embodiment of the needle output mechanism in FIGS. 1 and 2;

FIG. 5 is an exploded perspective view from another viewing angle of the preferred embodiment of the needle output mechanism in FIGS. 1 and 2;

FIG. 6 is a sectional side view of the preferred embodiment of the needle output mechanism cutting along line 1-1 in FIG. 2;

FIG. 7 is a sectional side view of the preferred embodiment of the needle output mechanism cut across line 2-2 in FIG. 2;

FIG. 8 is a sectional side view of the preferred embodiment of the needle output mechanism in FIGS. 1, 2, and 4 to 5, showing the needle output mechanism without a safety cover and a safety bolt;

FIG. 9 is an operational view of the preferred embodiment of the needle output mechanism in FIG. 8, showing a needle output operation assembly being pressed and driving a needle set located in a connecting member to protrude out of a base with a hard needle mounted in a soft needle;

FIG. 10 is an operational view of the preferred embodiment of the needle output mechanism in FIG. 9, showing the needle output operation assembly fixed in the base, and a needle base detached from the needle output operation assembly to drive the hard needle to detach from the soft needle;

FIG. 11 is an operational view of the preferred embodiment of the needle output mechanism in FIG. 9, showing a needle retraction operation member driving the soft needle to retract after injection;

FIG. 12 is an operational view (1) of the preferred embodiment of the needle output mechanism in FIG. 10, showing the needle output mechanism cutting a diameter reducing segment of a connecting head segment of the connecting member during retracting the needle set; and

FIG. 13 is an operational view (2) of the preferred embodiment of the needle output mechanism in FIG. 10, showing the needle output mechanism cutting the diameter reducing segment of the connecting head segment of the connecting member during retracting the needle set.

### DETAILED DESCRIPTION OF THE PREFERRED EMBODIMENTS

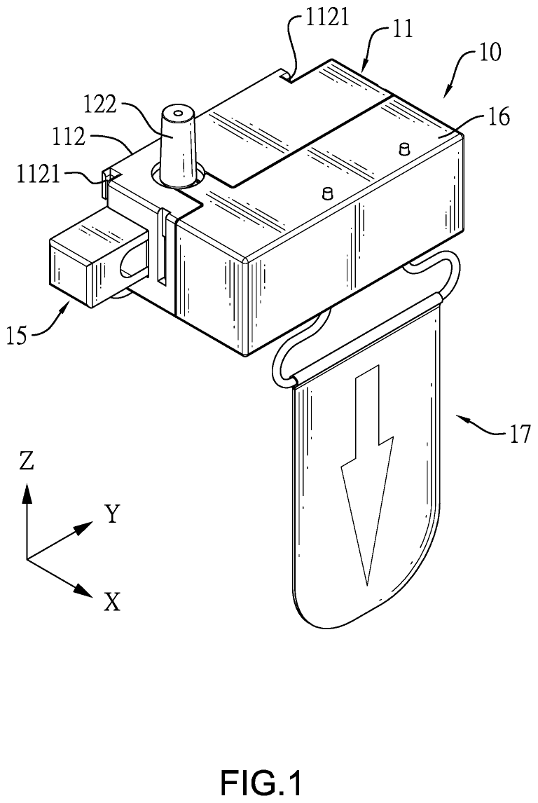

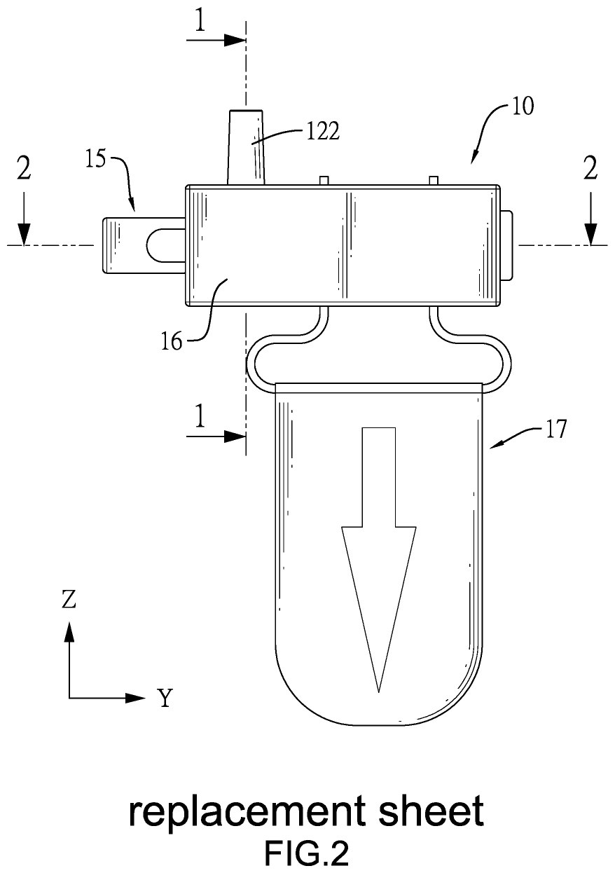

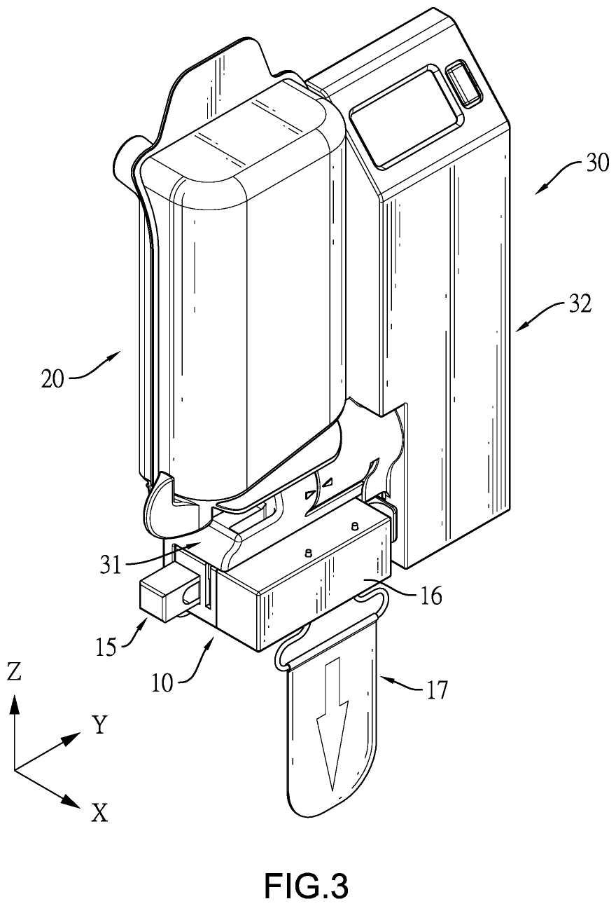

With reference to FIGS. 1 and 2, a preferred embodiment of a needle output mechanism 10 in accordance with the present invention is disclosed. With reference to FIG. 3, the needle output mechanism 10 is connected to a pharmaceutical agent container 20 and a drive module 30. The pharmaceutical agent container 20 contains a pharmaceutical agent. The drive module 30 is manual or electrical and processes human body injections via the needle output mechanism 10.

With reference to FIG. 3, the drive module 30 has an adapter mechanism 31 and an electrical driving mechanism 32. The adapter mechanism 31 is connected to the pharmaceutical agent container 20 and the electrical driving mechanism 32. The needle output mechanism 10 is connected to the adapter mechanism 31 of the drive module 30 and forms an injector. The adapter mechanism 31 has an input adapter, an output adapter, and a driving piston assembly. The input adapter is connected to the pharmaceutical agent container 20. The output adapter is connected to the needle output mechanism 10. The driving piston assembly is connected to the electrical driving mechanism 32. The drive module 30 draws the pharmaceutical agent from the pharmaceutical agent container 20 and then supplies the pharmaceutical agent to the needle output mechanism 10 and then the needle output mechanism 10 injects the pharmaceutical agent to the human body. The configuration is not limited to the above, as the needle output mechanism 10 can be assembled with other parts to meet the needs of different injectors.

With reference to FIGS. 1 and 2, in order to accurately illustrate the structure of the needle output mechanism 10 in a three-dimensional space, an X axis, a Y axis, and a Z axis are defined. The needle output mechanism 10 has a front side and a back side opposite to each other and arranged along the X axis, and the back side is used for contacting the human body.

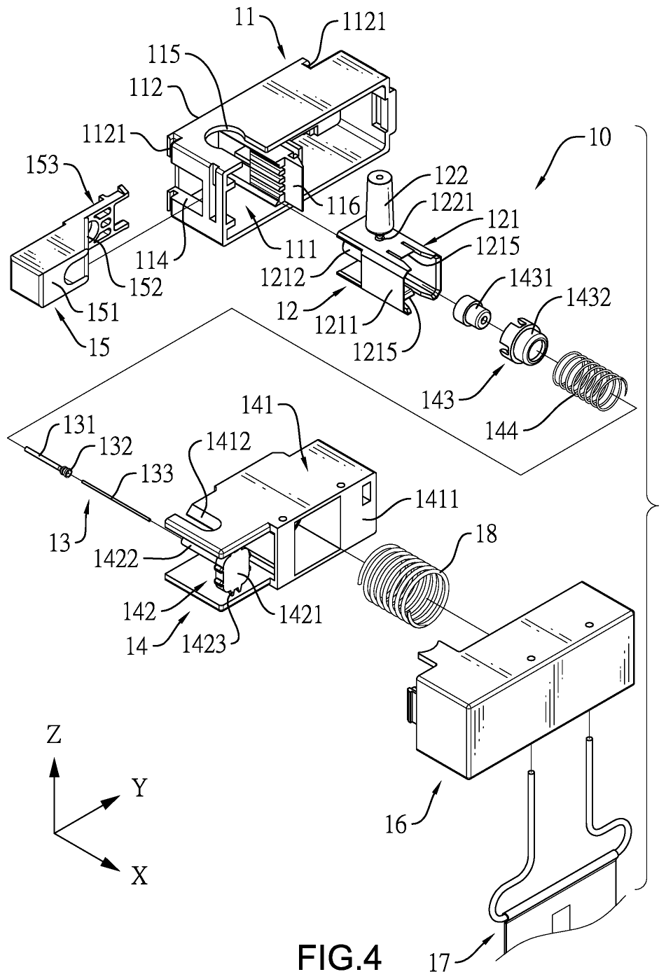

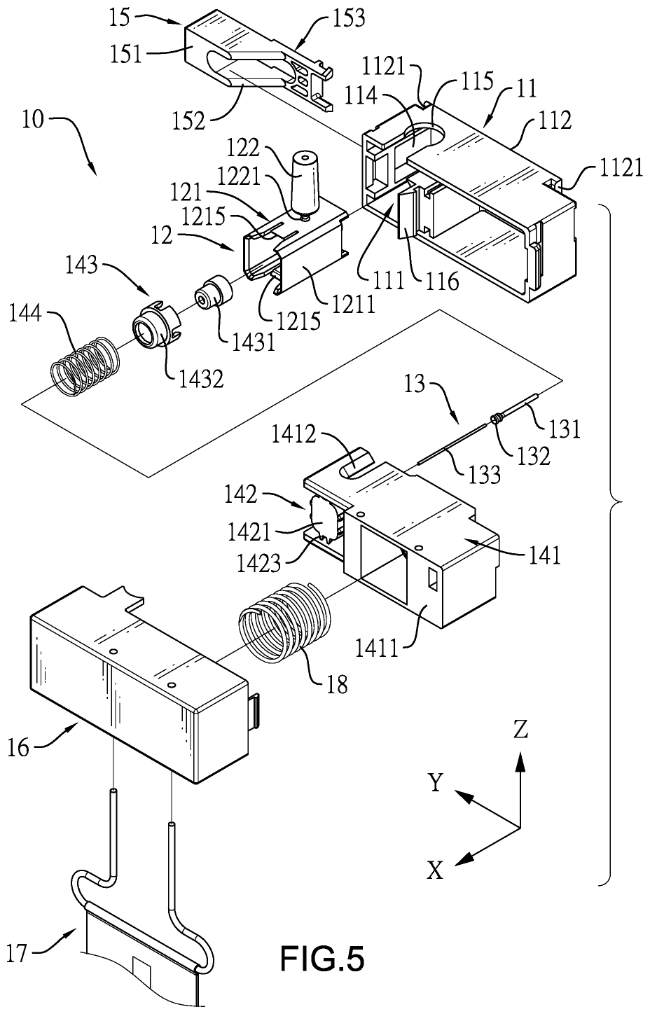

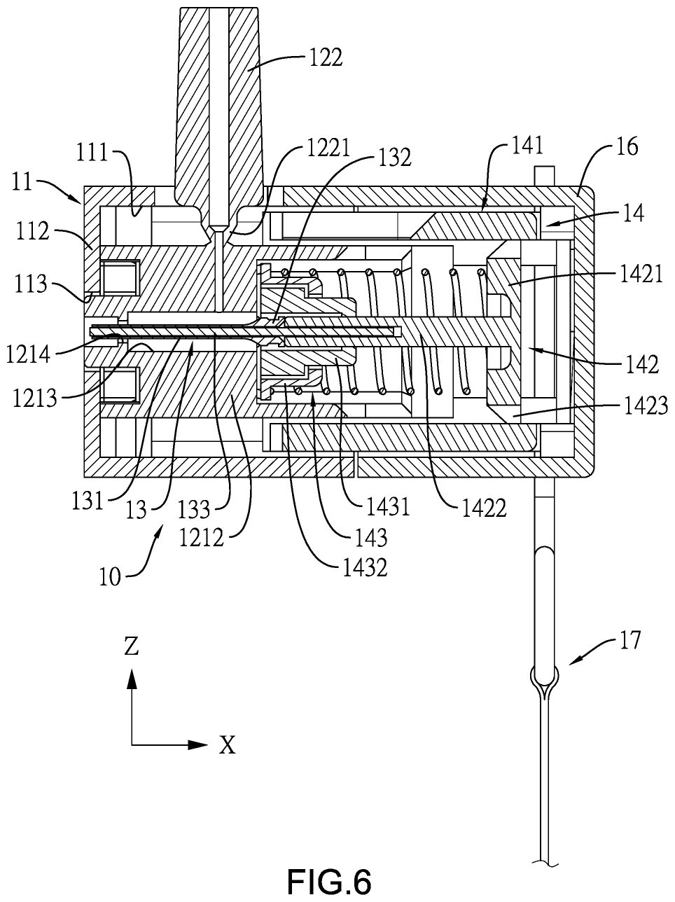

With reference to FIGS. 1, 2, and 3 to 6, the needle output mechanism 10 has a base 11, a connecting member 12, a needle set 13, a needle output operation assembly 14, and a needle retraction operation member 15.

With reference to FIGS. 1, 2, and 3 to 6, the base 11 is used for contacting human skin, and is connected to the drive module 30. In this preferred embodiment, the base 11 has an action room 111 having an opening facing a same direction as the front side on the X axis. The action room 111 has a base board 112 located on a same direction as the back side on the X axis. The base board 112 has a penetration hole 113 connected to the action room 111. The base 11 has a through hole 114 located on a side of the action room 111 along the Z axis. The through hole 114 is connected to the action room 111. The base 11 has an action opening 115 located on another side of the action room 111 along the Z axis. The action opening 115 is connected to the action room 111. The base 11 has a fixing buckling hook 116 located outside the action room 111.

With reference to FIGS. 1 and 3 to 4, the base 11 may further have two assembling segments 1121 respectively disposed on two sides, which are defined along the Y axis, of the base board 112 on the back side, and is assembled with the adapter mechanism 31 via the assembling segments 1121.

With reference to FIGS. 1, 2 and 3 to 6, the connecting member 12 is moveably mounted in the action room 111 of the base 11, and has a connecting base segment 121 and a connecting head 122. The connecting base segment 121 has a base annular wall 1211 and a needle set mounting segment 1212 mounted in the base annular wall 1211. The needle set mounting segment 1212 has a needle room 1213 and a needle plug hole 1214. The needle plug hole 1214 is connected to the needle room 1213 and is located on an end, which faces the base board 112 of the base 11, of the needle set mounting segment 1212. The needle plug hole 1214 corresponds in position to the penetration hole 113 of the base board 112. The connecting head 122 is mounted on the connecting base segment 121 and is connected to the needle room 1213 of the needle set mounting segment 1212. The connecting head 122 extends along the Z axis. The connecting head 122 is connected to the output adapter of the adapter mechanism 31.

With reference to FIGS. 3 to 6, in this preferred embodiment, the base annular wall 1211 has at least one cutting blade segment 1215 located on a side, which is away from the base board 112 of the base 11, of the base annular wall 1211. The connecting head 122 forms a diameter reducing segment 1221 on an end, which is connected to the connecting base segment 121, of the connecting head 122. A diameter of the diameter reducing segment 1221 progressively reduces and the diameter reducing segment 1221 can be cut off. The connecting head 122 of the connecting member 12 is mounted through the action opening 115 of the base 11.

With reference to FIGS. 3 to 6, the needle set 13 is mounted in the needle set mounting segment 1212 of the connecting member 12 and is capable of moving along the X axis. The needle set 13 can be operated to protrude needles and retract needles. The needle set 13 has a soft needle 131, a needle plug 132, and a hard needle 133. The soft needle 131 is mounted in the needle room 1213 of the needle set mounting segment 1212 and is capable of moving along the X axis. An end of the soft needle 131 points toward the needle plug hole 1214 and another end of the soft needle 131 is fixed with the needle plug 132. When the soft needle 131 is driven to protrude out of the base board 112 of the base 11, the needle plug 132 is stuck at the needle plug hole 1214. The hard needle 133 can be mounted in the needle plug 132 and the soft needle 131. The hard needle 133 is capable of protruding along with the soft needle 131. After the soft needle 131 protrudes, the hard needle 133 can be detached from the soft needle 131, such that the soft needle 131 is connected to the adapter mechanism 31 via the needle room 1213 and the connecting head 122 for forming a pharmaceutical agent flow path. The soft needle 131 can be retracted to the base 11 along with the connecting member 12.

With reference to FIGS. 3 to 6, the needle output operation assembly 14 is mounted in the base 11 and is capable of driving the needle set 13 to protrude. In this preferred embodiment, the needle output operation assembly 14 has a needle output operating unit 141, a needle base 142, a sealing unit 143, and an elastic unit 144.

With reference to FIGS. 3 to 6, the needle output operating unit 141 is mounted in the base 11 and is capable of moving along the X axis. The needle output operating unit 141 can be buckled and fixed by the fixing buckling hook 116 of the base 11. The needle output operating unit 141 has a button segment 1411 and an accommodating room located on a side of the button segment 1411. The accommodating room corresponds in position to the action room 111 of the base 11. The needle output operating unit 141 has a cutting knife segment 1412 located on a side, which corresponds in position to the connecting head 122, of the needle output operating unit 141. The cutting knife segment 1412 can be operated to cut off the diameter reducing segment 1221 of the connecting head 122 of the connecting member 12.

With reference to FIGS. 3 to 6, the needle base 142 is mounted in the action room 111 of the needle output operating unit 141 and is connected to the needle output operating unit 141. After the needle set 13 protrudes, the needle base 142 is detached from the needle output operating unit 141. The needle base 142 has a needle base head end 1421 and a needle base connecting rod 1422. The needle base head end 1421 is connected to the needle output operating unit 141 and is located in the needle output operating unit 141. The needle base connecting rod 1422 is formed on the needle base head end 1421. The needle base connecting rod 1422 is connected to the hard needle 133 and is capable of pushing the needle plug 132 of the needle set 13.

With reference to FIGS. 3 to 6, in this preferred embodiment, the needle base 142 and the needle output operating unit 141 are formed integrally. A connecting segment 1423 is formed between an edge of the needle base head end 1421 and an inner wall of the accommodating room of the needle output operating unit 141. The connecting segment 1423 can be cut off. The cutting blade segment 1215 of the connecting member 12 in the base 11 can cut the connecting segment 1423 to separate the needle base 142 and the needle output operating unit 141.

With reference to FIGS. 3 to 6, the sealing unit 143 is assembled with the elastic unit 144, and the sealing unit 143 and the elastic unit 144 are mounted in the connecting member 12. The sealing unit 143 is mounted outside the needle set mounting segment 1212 and covers the needle room 1213. The sealing unit 143 is sleeved on and fits the needle base connecting rod 1422. The elastic unit 144 is sleeved on the needle base connecting rod 1422. Two ends of the elastic unit 144 are respectively connected to the needle base head end 1421 of the needle base 142 and the sealing unit 143. In this preferred embodiment, the sealing unit 143 has a sealing gasket 1431 and a sleeving cover 1432 sleeved on the sealing gasket 1431. The elastic unit 144 abuts the sleeving cover 1432. The sealing gasket 1431 is sleeved on and fits the needle base connecting rod 1422.

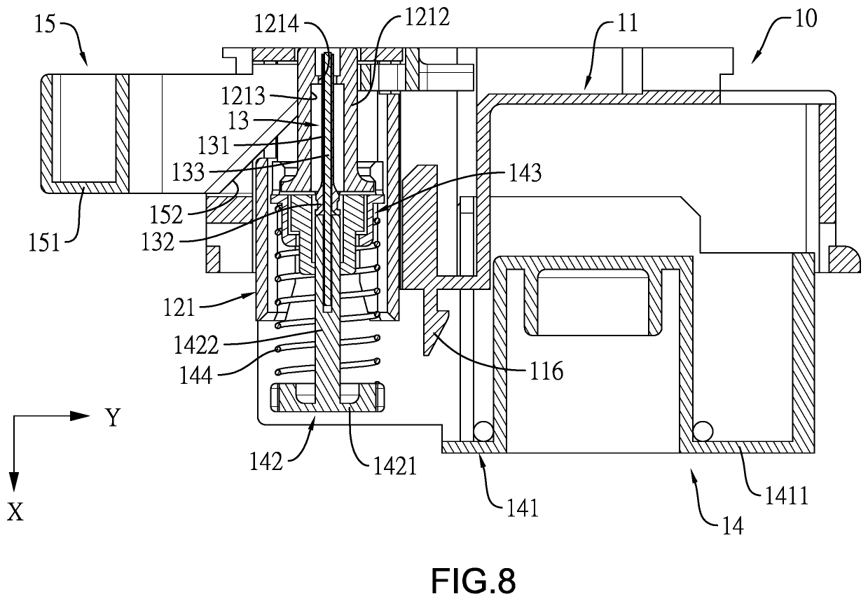

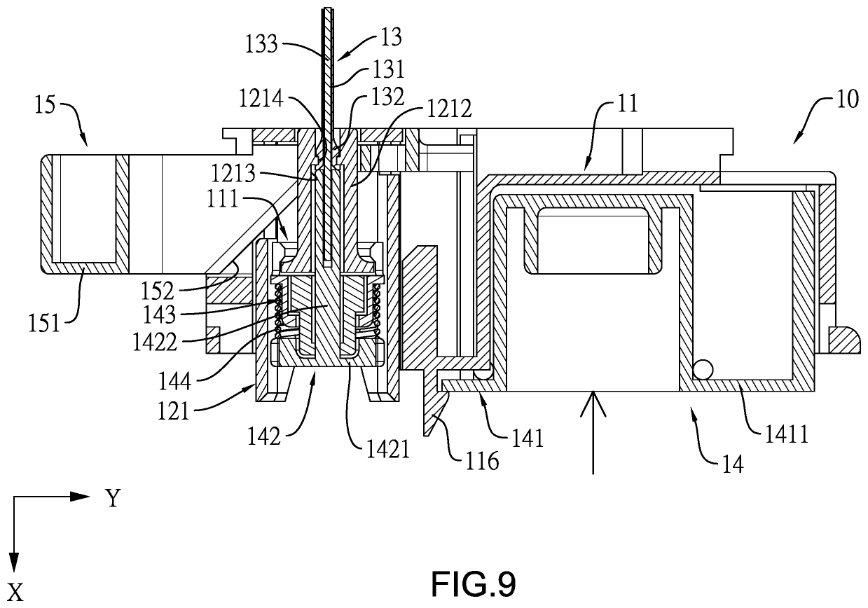

With reference to FIGS. 3 to 6, 8, and 9, when the needle output operating unit 141 is pressed toward the base 11, the hard needle 133 of the needle set 13 is mounted in the soft needle 131, and the needle output operating unit 141 drives the needle base 142 to push the needle set 13 to protrude out of the base board 112 of the base 11, and then the elastic unit 144 pushes the connecting member 12 to abut the base board 112 located on a back surface of the base 11, so the soft needle 131 and the hard needle 133, in an assembled configuration, pierce human skin. When the needle plug 132 is stuck at the needle plug hole 1214 of the needle set mounting segment 1212, the needle output operating unit 141 is buckled and fixed by the fixing buckling hook 116 of the base 11, and the connecting segment 1423 formed on the edge of the needle base 142 is cut off by the cutting blade segment 1215 of the connecting member 12. Then, the elastic unit 144 pushes out the needle base 142 and the needle base connecting rod 1422 of the needle base 142 pulls out the hard needle 133 from the soft needle 131, so the soft needle 131 is connected to the adapter mechanism 31 via the needle room 1213 of the connecting member 12 and the connecting head 122.

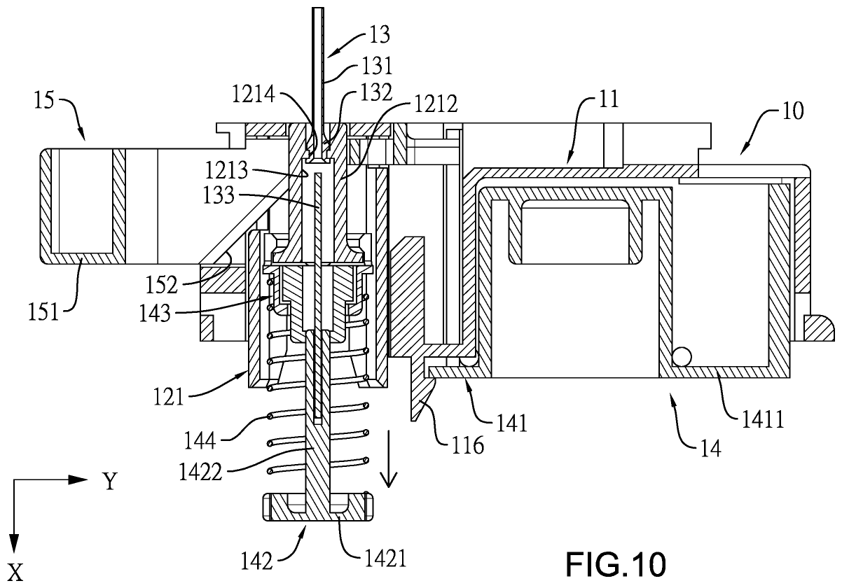

With reference to FIGS. 3 to 6, 9, and 10, the needle retraction operation member 15 extends through the through hole into the action room 111 from outside the base 11. The needle retraction operation member 15 has a pressing segment 151, an abutting inclined surface 152, and an extending segment 153. The pressing segment 151 is located outside the base 11. The abutting inclined surface 152 is located on a side, which extends into the action room 111, of the pressing segment 151. The abutting inclined surface 152 abuts the base annular wall 1211 of the connecting member 12. The extending segment 153 is located on the side, which extends into the action room 111, of the pressing segment 151. The extending segment 153 bypasses the needle set mounting segment 1212 of the connecting member 12. The extending segment 153 has a position limiting hook. The position limiting hook is limited by the base 11 such that the needle retraction operation member 15 can be mounted in the base 11 and is allowed to move within a limited distance without detaching from the base 11. When the pressing segment 151 of the needle retraction operation member 15 is pressed, the abutting inclined surface 152 pushes the connecting member 12 in the base 11 away from the base board 112 located on the back surface of the base 11, thereby retracting the connecting member 12 and the soft needle 131 of the needle set 13 that is mounted in the connecting member 12 back to the base 11.

With reference to FIGS. 3 to 6, the needle output mechanism 10 may additionally have a safety cover 16 and a safety bolt 17 for ensuring the safety of the needle output mechanism 10. The safety cover 16 covers the needle output operation assembly 14 and is capable of engaging with the base 11. The safety bolt 17 is mounted in the safety cover 16 and the needle output operating unit 141 of the needle output operation assembly 14, such that the needle output mechanism 10 can protrude the needle only after the safety bolt 17 is pulled off and the safety cover 16 is detached, thereby ensuring safety in use. Besides, a pushing elastic unit 18 is mounted between the safety cover 16 and the needle output operating unit 141, so the safety cover 16 will be automatically detached by the pushing elastic unit 18 after the safety bolt 17 is pulled off, thereby facilitating convenience in operation.

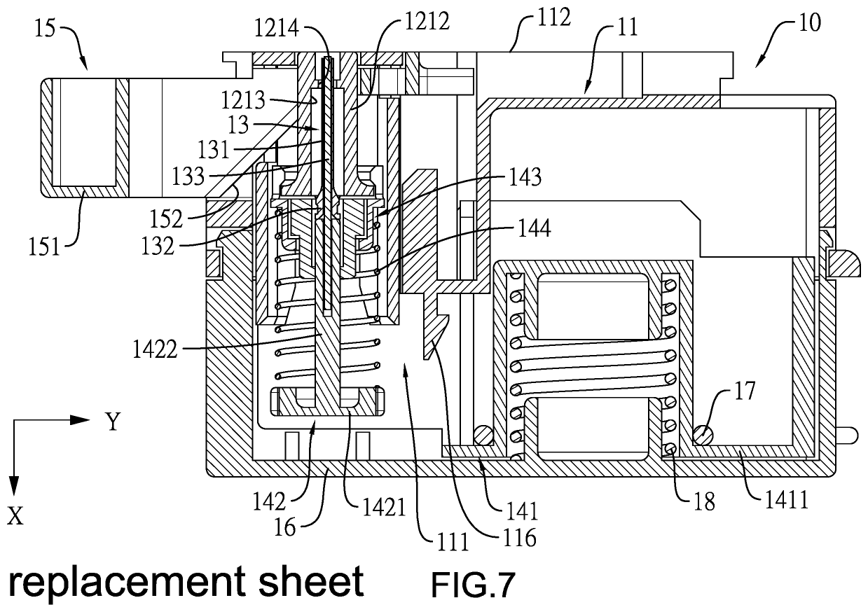

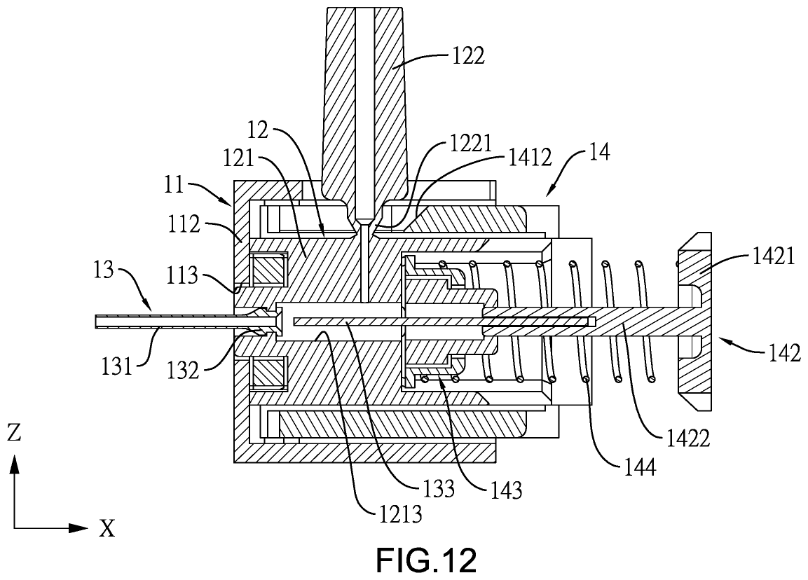

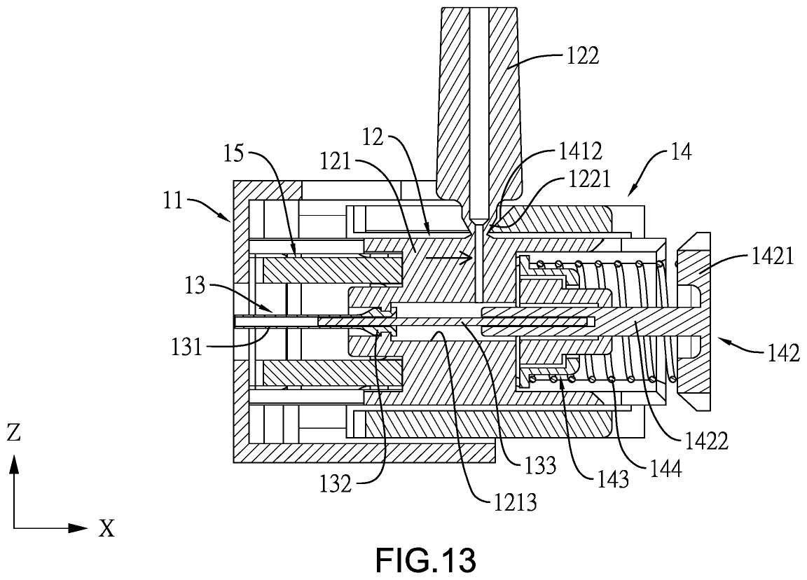

With reference to FIGS. 3 and 5 to 13, an application of the needle output mechanism in an electrical injector is disclosed. The adapter mechanism 31 of the drive module 30 is connected to the pharmaceutical agent container 20 and the electrical driving mechanism 32. The needle output mechanism 10 is connected to the adapter mechanism 31 of the drive module 30.

When a user intends to perform a subcutaneous injection, before the injection, the pharmaceutical agent container 20 and the adapter mechanism 31 of the drive module 30 are fixed at an intended injection site of a human body by pasting or bundling. The back surface of the base 11 of the needle output mechanism 10 is close to or contacts the human body. Then, the user pulls off the safety bolt 17 of the needle output mechanism 10 and detaches the safety cover 16, and then presses the needle output operating unit 141 of the needle output mechanism 10 to fix the needle output operating unit 141 on the base 11 and to push the needle set 13 via the needle base 142, so that the needle set 13 protrudes out of the base 11 with the hard needle 133 mounted in the soft needle 131 and directly pierces into the human body. After that, the cutting blade segment 1215 of the connecting member 12 cuts the connecting segment 1423 between the needle base 142 and the needle output operating unit 141, so the elastic unit 144 pushes the needle base 142 to pull out the hard needle 133 from the soft needle 131. The soft needle 131 is left in the human body and is connected to the adapter mechanism 31 via the connecting head 122 and the needle room 1213. Next, the user is allowed to operate the electrical driving mechanism 32 and drive the driving piston assembly of the adapter mechanism 31 to draw the pharmaceutical agent from the pharmaceutical agent container 20 and then supplies the pharmaceutical agent to the needle output mechanism 10 and injects the pharmaceutical agent into the human body via the soft needle 131. After injection, the user stops the electrical driving mechanism 32 and the driving piston assembly of the adapter mechanism 31. As a result, the injector is installed on the human body and the soft needle 131 stays piercing into the human body, so the user can process other timed injections via the electrical driving mechanism 32.

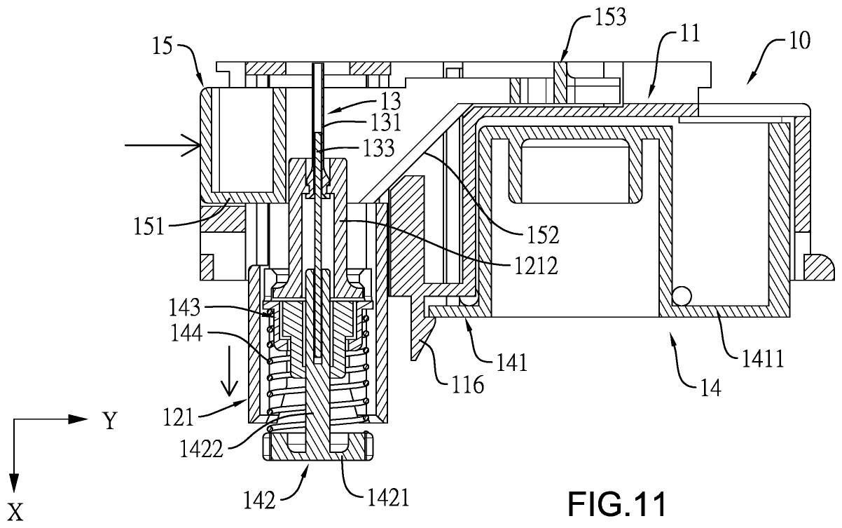

When the pharmaceutical agent in pharmaceutical agent container 20 is used up, with reference to FIGS. 11 to 13, the user presses the needle retraction operation member 15 of the needle output mechanism 10, uses the abutting inclined surface 152 of the needle retraction operation member 15 to push the connecting member 12 in the base 11 away from the base board 112 located on the back surface of the base 11, and pulls the soft needle 131 of the needle set 13 mounted in the connecting member 12 to retract to the base 11. At the same time, when the abutting inclined surface 152 of the needle retraction operation member 15 is pushing the connecting member 12, the cutting knife segment 1412 of the needle output operating unit 141 mounted on the base 11 cuts off the diameter reducing segment 1221 of the connecting head 122 of the connecting member 12 connected to the adapter mechanism 31, such that the needle output mechanism 10 cannot be reused, thereby ensuring the safety of using the needle output mechanism 10.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

1. A needle output mechanism comprising:
a base;
a connecting member moveably mounted in the base;
a needle set moveably mounted in the connecting member and having a soft needle, a needle plug mounted on an end of the soft needle, and a hard needle being capable of being mounted in the soft needle;
a needle output operation assembly moveably mounted in the base and connected to the needle set; the needle output operation assembly being capable of being operated to drive the needle set located in the connecting member to protrude out of the base with the hard needle mounted in the soft needle, and then drive the hard needle to detach from the soft needle; and
a needle retraction operation member moveably mounted in the base; the needle retraction operation member being capable of being operated to drive the connecting member and the soft needle in a needle-output state to retract back to the base.

2. The needle output mechanism as claimed in claim 1, wherein the base has an action room; a back surface of the base has a base board and a penetration hole formed on the base board and connected to the action room; a side of the base has an action opening connected to the action room; and a fixing buckling hook is mounted on the base and located outside the action room;
the connecting member is moveably mounted in the action room of the base; the connecting member has a connecting base segment and a connecting head; the connecting base segment has a base annular wall and a needle set mounting segment mounted in the base annular wall; the needle set mounting segment has a needle room formed in the needle set mounting segment and a needle plug hole formed on an end, which faces toward the base board, of the needle set mounting segment and connected to the needle room; the needle plug hole corresponds to the penetration hole of the base board; the connecting head is mounted on the connecting base and connected to the needle room in the needle set mounting segment; the connecting head is mounted through the action opening of the base; the base annular wall has at least one cutting blade segment;
the soft needle of the needle set is moveably mounted in the needle room of the needle set mounting segment; the needle plug that is mounted on the soft needle is capable of being fixed in the needle plug hole; and
the needle output operation assembly has a needle output operating unit, a needle base, a sealing unit, and an elastic unit; the needle output operating unit is moveably mounted in the base, and the needle output operating unit is capable of being fixed and buckled by the fixing buckling hook of the base; the needle output operating unit having a button segment and an accommodating room located on a side of the button segment; the accommodating room corresponds to the action room; the needle base is located in the accommodating room of the needle output operating unit and is connected to the needle output operating unit; the needle base has a needle base head end and a needle base connecting rod; a connecting segment is mounted between a periphery of the needle base head end and an inner wall of the accommodating room and is capable of being cut by the cutting blade segment; the needle base connecting rod is connected to the hard needle and is capable of pushing the needle plug of the needle set; the sealing unit assembled with the elastic unit is located in the connecting member; the sealing unit is located outside of the needle set mounting segment and covers the needle room; the sealing unit is tightly sleeved on an outer side of the needle base connecting rod; the elastic unit is sleeved on the outer side of the needle base connecting rod; two ends of the elastic unit are respectively connected to the needle base head end of the needle base and the sealing unit.

3. The needle output mechanism as claimed in claim 2 wherein the needle base and the needle output operating unit are formed integrally.

4. The needle output mechanism as claimed in claim 2 wherein a diameter reducing segment is formed on an end, which is connected to the connecting base segment, of the connecting head of the connecting member, is formed with a reduced diameter and is capable of being cut off; a cutting knife segment is mounted on a side, which corresponds to the connecting head, of the needle output operating unit; the cutting knife segment is capable of being operated to cut off the diameter reducing segment of the connecting head of the connecting member.

5. The needle output mechanism as claimed in claim 2 wherein a through hole is formed on a side, which is adjacent to the action room, of the base and is connected to the action room; the needle retraction operation member extends into the action room from an outer side of the base via the through hole; the needle retraction operation member has a pressing segment, an abutting inclined surface and an extending segment; the pressing segment extends to the outer side of the base; the abutting inclined surface is located on a side, which extends into the action room, of the pressing segment; the abutting inclined surface abuts the base annular wall of the connecting member; the extending segment is located on the side, which extends into the action room, of the pressing segment and bypassing the needle set mounting segment of the connecting member.

6. The needle output mechanism as claimed in claim 5 wherein an end, which is connected to the connecting base segment, of the connecting head of the connecting member, is formed with a reduced diameter and is capable of being cut off; a cutting knife segment is mounted on a side, which corresponds to the connecting head, of the needle output operating unit; the cutting knife segment is capable of being operated to cut off the diameter reducing segment of the connecting head of the connecting member.

7. The needle output mechanism as claimed in claim 2 the needle output mechanism has a safety cover and a safety bolt; the safety cover covers an outer side of the needle output operation assembly and is capable of engaging with the base; the safety bolt is mounted in the safety cover and the needle output operating unit of the needle output operation assembly.

8. The needle output mechanism as claimed in claim 7 characterized in that a pushing elastic unit is mounted between the safety cover and the needle output operating unit.

9. The needle output mechanism as claimed in claim 4, wherein the needle output mechanism has a safety cover and a safety bolt; the safety cover covers an outer side of the needle output operation assembly and is capable of engaging with the base; the safety bolt is mounted in the safety cover and the needle output operating unit of the needle output operation assembly.

10. The needle output mechanism as claimed in claim 5, wherein the needle output mechanism has a safety cover and a safety bolt; the safety cover covers an outer side of the needle output operation assembly and is capable of engaging with the base; the safety bolt is mounted in the safety cover and the needle output operating unit of the needle output operation assembly.

11. The needle output mechanism as claimed in claim 6, wherein the needle output mechanism has a safety cover and a safety bolt; the safety cover covers an outer side of the needle output operation assembly and is capable of engaging with the base; the safety bolt is mounted in the safety cover and the needle output operating unit of the needle output operation assembly.

12. The needle output mechanism as claimed in claim 7 characterized in that a pushing elastic unit is mounted between the safety cover and the needle output operating unit.

13. The needle output mechanism as claimed in claim 8 characterized in that a pushing elastic unit is mounted between the safety cover and the needle output operating unit.

14. The needle output mechanism as claimed in claim 9 characterized in that a pushing elastic unit is mounted between the safety cover and the needle output operating unit.

15. The needle output mechanism as claimed in claim 10 characterized in that a pushing elastic unit is mounted between the safety cover and the needle output operating unit.

16. The needle output mechanism as claimed in claim 11 characterized in that a pushing elastic unit is mounted between the safety cover and the needle output operating unit.
